(19) United States
(12) Patent Application Publication
(10) Pub. No.: US 2025/0387436 A1
(43) Pub. Date: Dec. 25, 2025

(54) SYSTEM AND METHOD FOR EXTRACTING GUT MICROBIOTA, CLASSIFYING THEIR DESIRABLE & UNDESIRABLE TRAITS, GENETICALLY MODIFYING THEM FOR DESIRED TRAITS & RESEEDING THEM FOR VARIOUS TREATMENTS & OUTCOMES

(71) Applicant: Srija Chaudhuri, Ambler, PA (US)

(72) Inventor: Srija Chaudhuri, Ambler, PA (US)

(21) Appl. No.: 18/752,344

(22) Filed: Jun. 24, 2024

(51) Int. Cl. A61K 35/74; C12N 9/22; C12N 15/11; C12Q 1/02

(52) U.S. Cl. CPC A61K 35/74; C12N 9/22; C12N 15/11; C12Q 1/02; C12N 2310/20

(57) ABSTRACT

Methods and systems for achieving genetically modified autologous microbiota transplant. Many health issues are attributed to the biomolecules produced by the gut microbiota. Transplanting someone else's microbiota with desirable traits is often rejected by the body's immune system. The novel method is to perform an autologous transplant by extracting and genetically modifying the microbiota of an individual and reimplanting the genetically modified microbiota into the same individual. This is expected to reduce if not eliminate adverse immune responses toward the genetically modified microbiota.

### TECHNICAL FIELD

The present disclosure is directed towards extracting samples of an individual's microbiome with the intention to genetically modify microorganisms for desirable traits. This genetically modified autologous microbiome flora is then reseeded into the individual's gut.

### BACKGROUND

Problems with the gut microbiome are often cited for multiple disorders and diseases including neurological disorders, metabolic diseases, gastrointestinal disorders, and certain cancers. In order to obtain desirable gut microbiome flora, microorganisms are extracted from a donor's fecal matter and seeded within the patient's gut. However, due to various bodily factors, including immune system defenses, these foreign microbiome florae are unable to survive in another host's body. A genetically modified autologous microbiome can have better survivability and acceptance within the body.

### SUMMARY

This specification describes techniques for seeding gut microbiota for better survivability and acceptance within the host body. The premise of seeding foreign microbiota is the host's gut microbiota either being unable to create certain biomolecules during the digestive process or creating too much of certain biomolecules, leading to health issues for the host individual. Dietary patterns and environmental factors have a significant effect on shaping gut microbiota. The native microbiota has a reason and a history for their mix and composition within the host body. According to Rinninella et al., each human's gut microbiota is shaped in early life as their composition depends on infant transitions (birth gestational date, type of delivery, methods of milk feeding, weaning period) and external factors, such as antibiotic use. This personal and healthy core native microbiota remain relatively stable in adulthood but differ between individuals due to enterotypes, body mass index (BMI) level, exercise frequency, lifestyle, and cultural and dietary habits. Accordingly, there is not a unique optimal gut microbiota composition since it is different for each individual. However, a healthy host-microorganism balance must be respected in order to optimally perform metabolic and immune functions and prevent disease development. If a foreign microbiota is seeded into a host, the long- and short-term survivability of the foreign microbiota is not guaranteed.

The genetic makeup of the microbiota as well as the expression of these genes play a role in the way the food is digested within the host body. The composition of the microbiota includes bacteria, viruses, fungi, and parasites. The human gastrointestinal (GI) tract contains an abundant and diverse microbial community and contains more than 100 trillion microorganisms. The density of bacterial cells in the colon has been estimated at 1011 to 1012 per milliliter. The gut microbiome encodes over 3 million genes producing thousands of metabolites, whereas the human genome consists of approximately 23,000 genes. For years, scientists have been interested in gut microbiota, but one of the major difficulties in the relevant research has been the ability to culture these microorganisms. During the last few years, new technologies have allowed researchers to phylogenetically identify and/or quantify the components of the gut microbiota by analyzing nucleic acids (DNA and RNA) directly extracted from stools. The majority of these techniques are based on the extraction of DNA and the amplification of the 16S ribosomal RNA gene (rRNA).

The human gut microbiome produces a functional complex of biomolecules, including nucleic acids, (poly) peptides, structural molecules, and metabolites. This impacts human physiology in multiple ways, especially by triggering inflammatory pathways in disease. The identification of various biomolecules released by the microbiota during the digestive process can play a crucial role in identifying the balance of the gut microbiome and identify how each plays a role in prevention and cure of various diseases.

The identification of one or more DNA signatures that are responsible for the secretion of the functional complex of biomolecules can help in correlating the biomolecules with a specific set of microbiotas. A change in the composition and behavior of the microbiota can modify the biomolecules released by the gut microbiome.

The composition of the microbiota is shaped by early life as well as other external factors, including diet and lifestyle. These are entrenched factors and as a result make the composition of the microbiota similarly entrenched. Inserting new flora into the microbiota may or may not change the composition of the microbiota, making the biomolecules secreted by them equally hard to change at least for the long term.

Modifying the microbiota DNA and/or the expression of the DNA can lead to changes in the behavior of the microbiota. These changes can be orchestrated in such a way that the biomolecules secreted by the microbiota are also changed to ensure that a desirable composition of the biomolecules within the gut is achieved.

The treatment process starts with extracting samples of the gut microbiome from the person to be treated. Then, a select group of microbiota organisms are identified for gene modification based on their role in release of various biomolecules. The type of genetic modification will depend upon the type of biomolecule change desired.

The genetic modification can be done using various techniques, such as CRISPR (Clustered Regularly Interspaced Short Palindromic Repeats), electroporation linear DNA, recombinant suicide plasmid, etc. Over-expression of the desired gene can be achieved via plasmid-mediation.

After undergoing gene modification, the modified microbiota is tested in a laboratory for the desired biomolecule secretion and composition. The testing is conducted such that the conditions of the gut are simulated. Testing is also conducted to ensure no undesirable biomolecules that may harm the patient are released.

To increase safety measures, an additional kill switch gene may also be introduced into the genetically modified microbiota. The kill switch will enable the microbiota to be eliminated from the patient's microbiome using a targeted antibiotic medication. This will ensure that if any undesirable effect is observed, the genetically modified autologous microbiota transplant can be reversed.

After the determination of the safety of the genetically modified autologous microbiota, the microbiota is transplanted into the patient. The transplantation can be done at various locations within the stomach, small intestine, and large intestine.

After transplantation, the patient's response to the autologous transplant of genetically modified microbiota is observed. If the treatment response is as per the expected parameters, the transformed gut microbiome flora can be a permanent component of the patient's microbiome. If at any time an adverse effect is observed due to the microbiota transplant, the genetically modified microbiota can be rolled back using the targeted antibiotics that use the kill-switch gene modification to kill the genetically modified microorganisms.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 is an illustrative diagram of gut microbiome composition unique to each individual. It also shows that the natural gut microbiota can be saved by freezing for future use.

FIG. 2 shows the workflow of extracting an individual's microbiota, performing gene modification on the microbiota, testing the modified microbiota for desirability, and reimplanting the genetically modified autologous microbiota into the respective individual.

FIG. 3 shows the workflow of fine-tuning genetically modified microbiota by further modifying their genes for improved results.

FIG. 4 shows the workflow for reversing the genetically modified microbiota to the original natural microbiome state by using targeted antibiotics to kill genetically modified microbiota and reimplanting of the original natural microbiota.

### DETAILED DESCRIPTION

According to an aspect of the present disclosure, systems and methods for transplant of genetically modified autologous gut microbiota are disclosed. Currently, microbiota transplant is performed by transplanting desirable microbiota from a donor into a recipient who has undesirable microbiota. This can be utilized for treatment of various conditions including neurological disorders, metabolic diseases, gastrointestinal disorders, certain cancers, etc. However, this process suffers from a high rate of failure due to recipient's immune system response, killing off transplanted donor microbiota. Thus, eliminating the desirable microbiota.

CAR T-cell therapy, also known as adoptive cell transfer, is a cancer immunotherapy treatment that uses a patient's T cells to treat their cancer. T cells are a type of immune system cell that are taken from the patient's blood and genetically altered in a lab to produce specialized receptors on their surface, called CARs. After a few weeks, the patient receives a drip containing the engineered T cells, which recognize and attack the cancer cells.

Combining the processes used in gut microbiota transplant and CAR T-cell therapy can help overcome the problem with recipient's immune system killing the donor's transplanted microbiota.

Genetically modifying patient's own microbiota for desirable effects and then transplanting the microbiota back into the patient can change the characteristics of patient's own microbiota into a desirable microbiome composition. Since patient's own microbiota is used for genetic modification, there is a very high chance that the patient's immune system will not attack the genetically modified autologous microbiota after it is transplanted back into the patient.

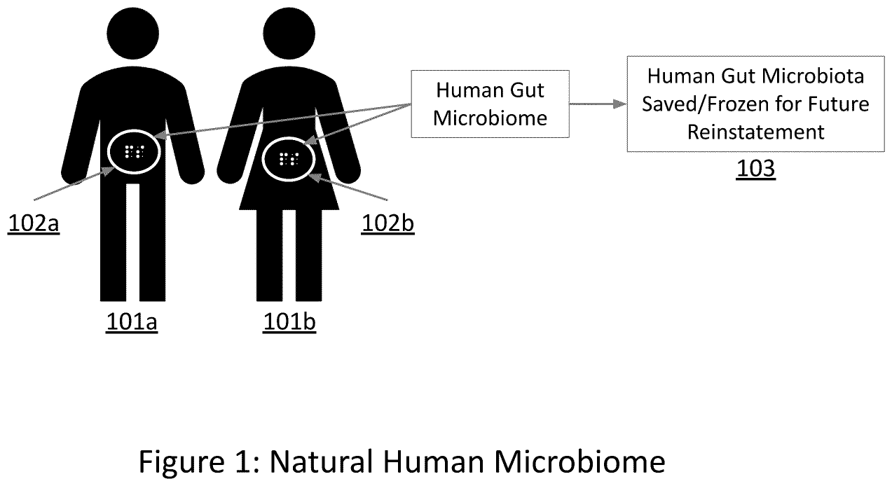

FIG. 1 describes the occurrence of natural human gut microbiome (102a, 102b). The human gut microbiome is highly diverse, with each person (101a, 101b) containing over 1,000 phylotypes, which are clusters of sequences with as much diversity as named species. The gut microbiome contains around 500-1,000 species of microbes, which have 100 times more unique genes than the human genome.

The gut microbiome's composition and diversity can vary across populations and over time. Some factors that can affect the gut microbiome include:
Diet: People who eat a variety of plant-based foods tend to have a more diverse gut microbiome.
Personality: People with larger social networks tend to have a more diverse gut microbiome, while anxiety and stress can lead to a reduced diversity.
Genetics: Identical twins only share about a third of their gut bacteria, which is only slightly more than two unrelated people.
Pet ownership: The gut microbiome of pet owners and those without pets is dominated by Firmicutes, but some operational taxonomic units (OTUs) are differentially abundant based on pet exposure.

The gut microbiome can affect many aspects of health and disease, including digestion, immunity, metabolism, development, and behavior.

Before embarking on changing the composition and behavior of one's microbiome, one can save their current microbiome (103). This can help in reversing the microbiome to its original composition in case any adverse event and/or side-effects are observed with the modified gut microbiota. The saving process may include extracting one's microbiota and then freezing it to preserve it for future use.

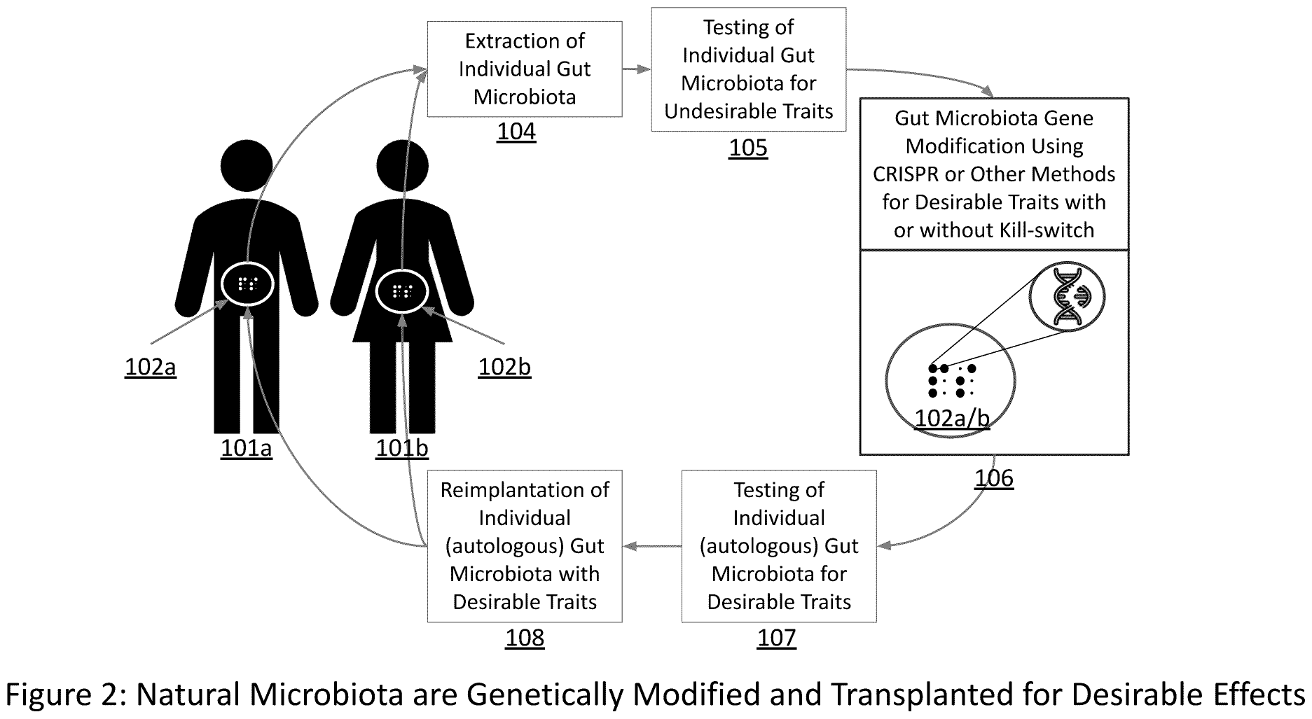

FIG. 2 describes the process of genetic modification of naturally occurring gut microbiota and transplanting it back into the patient for desirable effect. The process starts with the extraction of patient's naturally occurring gut microbiota (104).

The microbiota extracted from an individual can then be tested for desirable and undesirable traits (105).

In order to minimize the effect of undesirable traits, gene modification of the microbiota can be undertaken using gene modification techniques such as CRISPR (106). To have the ability to kill the genetically modified microbiota, an additional gene modification can be done on the microbiota. This gene modification will act as the kill switch for the microbiota in the presence of a specific antibiotic.

After gene modification of the microbiota, the microbiome is tested to confirm if the modified microbiota release the desirable biomolecules and doesn't release the undesirable biomolecules (107). This is to make sure the gene modification has the intended effect.

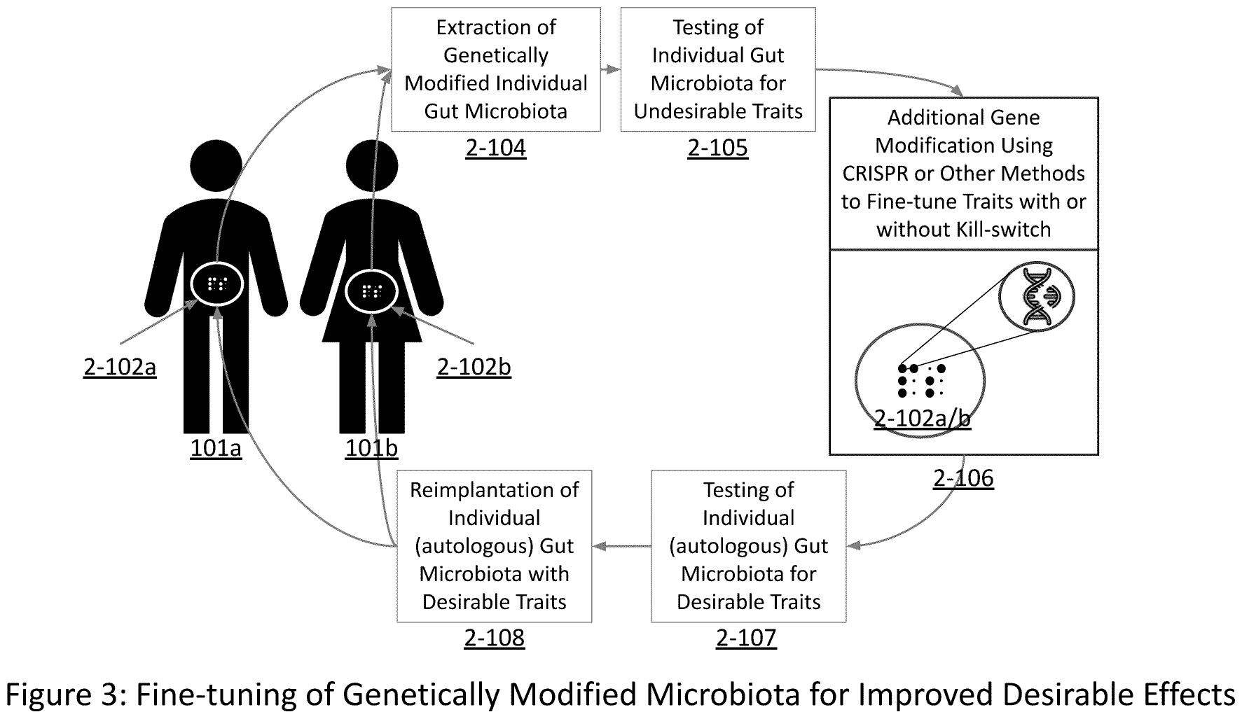

FIG. 3 describes the process of fine-tuning the genetic modification of gut microbiota that was previously genetically modified. This is performed to optimize the release of desirable biomolecules by the genetically modified gut microbiota.

The steps in FIG. 3 are similar to the steps in FIG. 2. The only difference between the two methods is the starting sample of the gut microbiota. In FIG. 2, the starting sample is the naturally occurring gut microbiota. However, in FIG. 3, the starting sample is the genetically modified gut microbiota that was previously genetically altered and implanted into the gut.

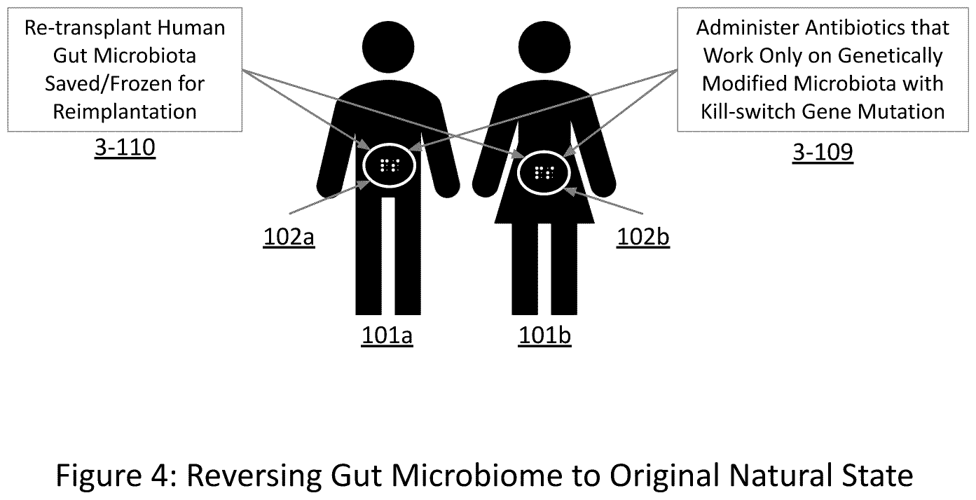

FIG. 4 describes the process of reversing the genetically modified gut microbiota transplant so that the original natural gut microbiome is reinstated. This may be necessary when the patient has experienced adverse effects due to the genetically modified gut microbiota, does not benefit from the treatment, or would like to reverse to the original natural microbiome due to a personal choice.

Reversing the genetically modified microbiome to its original natural state begins with administering the antibiotic that works only on the genetically modified microbiota with the kill-switch gene mutation (3-109).

After elimination of the genetically modified gut microbiota, the previously saved/frozen natural microbiota are transplanted back into the patient (3-110). After the elimination of the genetically modified gut microbiota, it is easier for the reimplanted natural microbiota to take hold and flourish in the gut.

1. Microbiota extraction for gene modification obtained from:
human gut from one or more sites of the gut;
by one or multiple means of extraction;
genetically modifying gut microbiota;
genetically modified microbiota implantation into:
human gut in one or more sites of the gut;
by one or multiple means of implantation;
thus, achieving genetically modified autologous microbiota transplant.

2. The gene modification and transplantation method of claim 1, further comprising:
testing of biomolecules produced by the extracted microbiota for one or multiple undesirable traits.

3. The gene modification and transplantation method of claim 1, further comprising:
testing of biomolecules produced by the extracted microbiota for one or multiple desirable traits.

4. The gene modification and transplantation method of claim 1, further comprising:
culture of extracted microbiota by one or multiple means.

5. The gene modification and transplantation method of claim 1, further comprising:
culture of extracted microbiota for one or multiple times.

6. The gene modification and transplantation method of claim 1, further comprising:
gene modification of extracted or cultured microbiota to enhance one or more desirable traits.

7. The gene modification and transplantation method of claim 1, further comprising:
gene modification of extracted or cultured microbiota to suppress one or more undesirable traits.

8. The gene modification and transplantation method of claim 1, further comprising:
testing of biomolecules produced by the genetically modified microbiota for one or multiple desirable traits.

9. The gene modification and transplantation method of claim 1, further comprising:
testing of biomolecules produced by the genetically modified microbiota for one or multiple undesirable traits.

10. The gene modification and transplantation method of claim 1, further comprising:
rollback of the genetically modified microbiota to reinstate the original natural microbiome.

11. The gene modification and transplantation method of claim 1, further comprising:
gene modification of extracted or cultured microbiota to embed a kill-switch so that the transplanted microbiota can be killed using a specific antibiotic.
